(19) United States
(12) Patent Application Publication
(10) Pub. No.: US 2015/0073342 A1
(43) Pub. Date: Mar. 12, 2015

(54) Linearly Stationary Catheter Drive Assemblies For Remote Catheter Positioning Systems

(71) Applicant: Catheter Robotics Inc., Budd Lake, NJ (US)

(72) Inventors: Robert Pacheco, Bayside, NY (US); Steve Foley, Kerrville, TX (US); David Jenkins, Budd Lake, NJ (US); Thomas Jackson, Cambridge (GB); Luke Hares, Milton (GB)

(21) Appl. No.: 14/478,023

(22) Filed: Sep. 5, 2014

Related U.S. Application Data
(60) Provisional application No. 61/874,446, filed on Sep. 6, 2013.

(51) Int. Cl. A61M 25/01

(52) U.S. Cl. CPC A61M 25/0113; A61M 2205/50; A61M 2205/33
USPC 604/95.04

(57) ABSTRACT

Systems, methods, and devices of the various embodiments provide linearly stationary catheter drive assemblies enabled to move a catheter's sheath along a linear axis while holding the catheter handle stationary along that linear axis. In the various embodiments, the linearly stationary catheter drive assembly may be configured to move the catheter's sheath along the linear axis while holding the catheter handle stationary along the linear axis and rotating the catheter handle about the linear axis. In an embodiment, a linearly stationary catheter drive assembly may include a loop drive configured to move the catheter sheath along the linear axis. In an embodiment, a linearly stationary catheter drive assembly may include a pinch drive configured to move the catheter sheath along the linear axis.

### CROSS REFERENCE TO RELATED APPLICATIONS

The present invention claims the benefit of priority to U.S. Provisional Patent Application No. 61/874,446, entitled “LINEARLY STATIONARY CATHETER DRIVE ASSEMBLIES FOR REMOTE CATHETER POSITIONING SYSTEMS,” filed Sep. 6, 2013, the entire contents of which are incorporated herein by reference.

### BACKGROUND

Many procedures involving catheter insertion, such as invasive electrophysiology procedures, rely on fluoroscopy or other radioactive imaging techniques to help navigate and position the catheter within a patient's body at a particular site, such as in the heart or inside a blood vessel in the circulatory system. High dosages of radiation can have long term adverse health effects. A patient may be directly exposed only once or twice to radiation during such procedures and avoid such adverse effects. However, physicians, medical technicians and staff can experience a large cumulative radiation dosage over time, both directly and indirectly, from conducting many procedures.

To protect the operator and staff from this radiation, shielding such as lead aprons, gowns, glasses, skirts, etc., is worn. Such lead clothing, especially a lead apron, is quite heavy and uncomfortable, and its use has been associated with cervical and lumbar spine injury.

### SUMMARY OF THE INVENTION

Systems, methods, and devices of the various embodiments provide linearly stationary catheter drive assemblies enabled to move a catheter's sheath along a linear axis while holding the catheter handle stationary along that linear axis. In the various embodiments, the linearly stationary catheter drive assembly may be configured to move the catheter's sheath along the linear axis while holding the catheter handle stationary along the linear axis and rotating the catheter handle about the linear axis. In an embodiment, a linearly stationary catheter drive assembly may include a loop drive configured to move the catheter sheath along the linear axis. In an embodiment, a linearly stationary catheter drive assembly may include a pinch drive configured to move the catheter sheath along the linear axis.

### BRIEF DESCRIPTION OF THE DRAWINGS

The accompanying drawings, which are incorporated herein and constitute part of this specification, illustrate exemplary embodiments of the invention, and together with the general description given above and the detailed description given below, serve to explain the features of the invention.

FIG. 1 and FIG. 2 are perspective views of a catheter drive assembly including a loop drive suitable for use in one or more embodiments.

FIG. 3A is a component diagram illustrating a catheter drive assembly as illustrated in FIG. 1 and FIG. 2 in one or more embodiments.

FIG. 3B is a component diagram illustrating a catheter drive assembly as illustrated in FIG. 1 and FIG. 2 in one or more additional or alternative embodiments.

FIGS. 4A-4C are diagrams illustrating relationships between rotational movement of an example loop drive and linear movement of a catheter sheath in one or more embodiments.

FIG. 5 is a perspective view of a pinch drive in one or more embodiments.

FIG. 6 is a cutaway perspective view of a pinch drive as illustrated in FIG. 5, in or more embodiments.

FIG. 7 is an exploded perspective view of a pinch drive as illustrated in FIG. 5 in one or more additional or alternative embodiments.

FIGS. 8A-8C are diagrams illustrating relationships between rotational movement of rollers of an example pinch drive and linear movement of a catheter sheath in one or more embodiments.

FIG. 9 is a perspective view of a catheter drive assembly including a pinch drive in one or more embodiments.

FIG. 10 is a system diagram illustrating an embodiment catheter positioning system.

### DETAILED DESCRIPTION

Various embodiments will be described in detail with reference to the accompanying drawings. Wherever possible, the same reference numbers will be used throughout the drawings to refer to the same or like parts. References made to particular examples and implementations are for illustrative purposes and are not intended to limit the scope of the invention or the claims.

Systems, methods, and devices of the various embodiments provide linearly stationary catheter drive assemblies enabled to move a catheter's sheath along a linear axis while holding the catheter handle stationary along that linear axis. In the various embodiments, the linearly stationary catheter drive assembly may be configured to move the catheter's sheath along the linear axis while holding the catheter handle stationary along the linear axis and rotating the catheter handle about the linear axis. Catheter drive assemblies according to the various embodiments may include a catheter driver and a catheter support coupled to the catheter driver. The catheter support may be configured to hold a handle of a catheter and the catheter driver may be configured to move a sheath of the catheter along a linear axis while the catheter support and the catheter handle remain stationary along the linear axis. In this manner, the catheter drive assemblies of the various embodiments may be linearly stationary in that the catheter drive assemblies of the various embodiments may enable a catheter sheath to be extended or retracted along the linear axis while the catheter support and the catheter handle do not move back or forth along that linear axis.

The linearly stationary catheter drive assemblies of the various embodiments may reduce the overall footprint of catheter positioning systems employing the embodiment linearly stationary catheter drive assemblies when compared with catheter positioning systems configured to move the catheter handle in a linear direction (e.g., track transiting catheter positioning systems) because the linearly stationary catheter drive assemblies may not require extensions approximately equal to the length of the catheter sheath in the linear direction along which to move the catheter handle. Additionally, the various embodiment linearly stationary catheter drive assemblies may accommodate catheters with longer sheaths without requiring modification (e.g., adding track length or additional sheath supports) to the linearly stationary catheter drive assemblies. In the various embodiments, the catheter contacting surfaces on the linearly stationary catheter drive assemblies may be sterile components, either sterilizable or disposable, to avoid introducing contaminants into the body of a patient.

In an embodiment, the catheter driver of a linearly stationary catheter drive assembly may be a loop drive configured to move the catheter sheath along the linear axis. In an embodiment, the loop drive may include a sheath guide wheel encircling the catheter support and configured to hold the shaft of the catheter around the outer edge of the sheath guide wheel. A guide wheel motor may rotate the shaft guide wheel, catheter support, and catheter handle together about an axis of rotation other than the linear axis, thereby extending or retracting the catheter shaft along the linear axis. In an embodiment, the guide wheel, catheter support, and catheter handle may be supported by a support frame which may be rotated by a frame motor, thereby rotating the support frame the guide wheel, catheter support, and catheter handle about the linear axis. In this manner, the catheter handle may rotate about the linear axis while not moving forward or backward along the linear axis. In an embodiment, the guide wheel motor may be supported by the support frame. In another embodiment, the guide wheel motor may be located in a portion of the catheter drive assembly not supported by the support frame.

In an embodiment, the catheter driver of a linearly stationary catheter drive assembly may be a pinch drive configured to move the catheter sheath along the linear axis. In an embodiment, the pinch drive may include a pair of rollers configured to be rotated by a roller motor in opposite directions to move the shaft of the catheter along the linear axis. In an embodiment, the pinch drive, catheter support, and catheter handle may be supported by a support frame. The pinch drive and catheter support may be rotationally coupled to the support frame, and a frame motor may rotate the pinch drive, catheter support, and catheter handle about the linear axis. In this manner, the catheter handle may rotate about the linear axis, approximately about the linear axis, or about a longitudinal axis of the catheter handle itself, which may or may not be in complete or precise alignment with the linear axis, while not moving forward or backward along the linear axis.

In an embodiment, a catheter positioning system may comprise a linearly stationary catheter drive assembly comprising a catheter driver and a catheter support configured to hold a handle of a catheter, a remote controller, and a processor connected to the remote controller and one or more motor of the catheter driver, the processor configured with processor-executable instructions to perform operations to activate one or more motors of the catheter driver to control in response to an input from the remote controller. In an embodiment, the catheter driver may be a loop drive. In another embodiment, the catheter driver may be a pinch drive.

Any type of catheter may be suitable for use with the various embodiments. Example catheters that may be used in various embodiments may include a handle portion and tube portion. The handle portion may be located at a proximal end of the catheters while the distal end of the tube portion may be inserted into the body of a patient. The handle portion of example catheters may also include an irrigation port, which may be used to introduce water or other fluids to lubricate the catheters and ease insertion or retraction into the patient. The handle portion may also include a back port through which one or more wires or cables may leave the handle portion. The one or more wires or cables may supply power to the example catheters or transmit signals, such as sending commands from a remote controller or other control device to the catheters or relaying data from one or more transducers present on the example catheters. Example catheters may include controls (e.g., on the handle portion) that control the behavior of the catheters. An example control that may be included on a catheter include a front flange and rear flange that may be squeezed together such that this motion may move one or more mechanism at the tip of the catheter (e.g., extending or retracting a laser tip from inside a tube portion of the catheter). The laser tip may be retracted by pulling the front flange and rear flange apart. Other example controls that may be include on a catheter include controls for deflecting the tip of the catheter to ease navigation inside a patient and/or for controlling one or more transducers at the tip (e.g., electrical leads, one or more sensor devices, ultrasound devices, etc.). The various embodiments may be applicable to catheters with different types of controls. The various embodiments may be applicable to catheters with different types of controls. The various embodiments may be especially applicable to flexible catheters, such as angioplasty catheters, but any type of catheter may be suitable for use with the various embodiments.

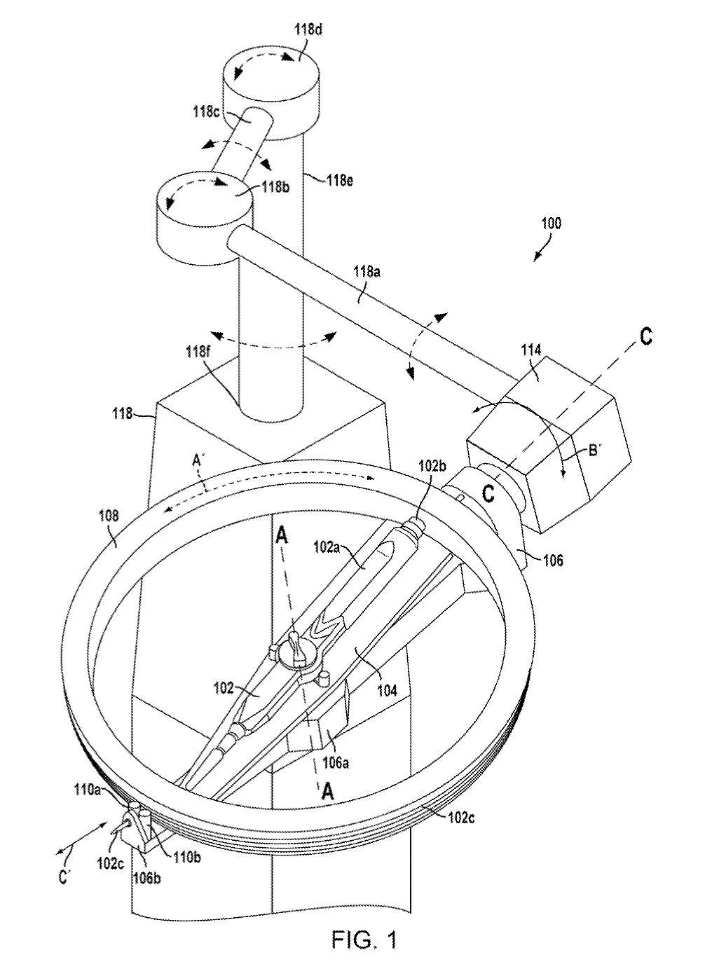

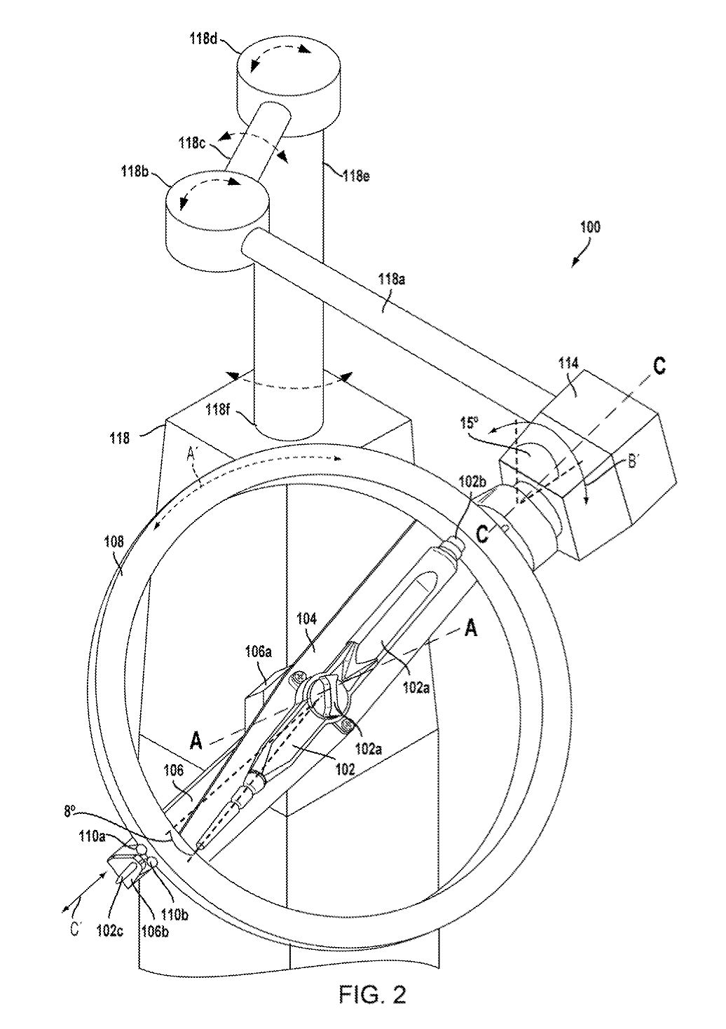

FIG. 1 and FIG. 2 illustrate a catheter drive assembly 100 including a loop drive according to one or more embodiments. FIG. 1 illustrates the sheath guide wheel 108, catheter support 104, and catheter handle 102a in a first position and FIG. 2 illustrates the sheath guide wheel 108, catheter support 104, and catheter handle 102a moved to a second position as discussed further below. The sheath guide wheel 108 may be a rotational component that translates a rotational movement of the catheter sheath to a linear movement of the catheter sheath. In some embodiments, the sheath guide wheel 108 may be sterile when in use, because it comes into contact with the catheter sheath. The guide wheel 108 may be removable so that after use it may be removed for disposal (i.e., the sheath guide wheel may be a single use component) or for sterilization prior to the next use.

Referring to FIG. 1, the catheter drive assembly 100 may be coupled to a base 118 including various articulating joints 118b, 118d, 118f, and arms 118a, 118c, and 118e. The base 118 may be coupled to a drive box 114 of the catheter drive assembly 100, such as through the arms 118a, 118c, and 118e. In an embodiment, connections for power, data, etc., to/from the catheter drive assembly 100 (e.g., motor control signals, motor power, catheter data connections, etc.) may pass through the base 118 to the drive box 114. In various embodiments, one or more of the arms 118a, 118c, and 118e may be rotated and one or more of the articulating joints 118b, 118d, and 118f may be positioned so as to affect the position or orientation of the catheter drive assembly 100. In an embodiment, the arm 118a of the base 118 may be rotationally coupled to the drive box 114 such that the angle of the catheter drive assembly 100 may be rotated about a longitudinal axis of the arm 118a. Other rotational movements of the arm portions of the base 118 (e.g., arms 118c, 118e) may also be possible, which may be capable of positioning the catheter drive assembly 100 in embodiments. The drive box 114 of the catheter drive assembly 100 may be rotationally coupled to a support frame 106 which may support a loop drive that includes the sheath guide wheel 108 and a guide wheel motor. The support frame 106 may extend along the linear axis C. The drive box 114 may include a frame motor configured to rotate the support frame 106 about the linear axis C clockwise or counterclockwise, such as in the B′ directions.

The catheter support 104 may be coupled to the sheath guide wheel 108 of the loop drive, and the sheath guide wheel 108 may encircle the catheter support 104. The support frame 106 may include a rotator housing 106a that may be coupled to and support the catheter support 104. The support frame 106 may be coupled to and support the catheter support 104, such as in a rotation plane above the support frame 106 through a rotating shaft extending from the center of the rotator housing 106a and coupling to the catheter support 104 at or near the axis of rotation A. In this manner, rotation of the rotating shaft in the rotator housing 106a may rotate the catheter support 104, the sheath guide wheel 108 coupled to it, and the catheter handle 102a together clockwise or counterclockwise in the A′ direction about the axis of rotation A.

The catheter support 104 may be configured to hold the catheter handle 102a of the catheter 102 and the catheter sheath 102c of the catheter 102 may extend through an opening in the sheath guide wheel 108 and loop around an outer edge, channel, groove, or other catheter sheath retaining surface of the sheath guide wheel 108. While the various examples are illustrated with the catheter sheath 102c looping around the sheath guide wheel 108 clockwise in the A′ direction, the catheter sheath 102c may loop around the sheath guide wheel 108 clockwise or counterclockwise in the A′ direction depending on how an operator of the catheter drive assembly 100 may thread the catheter sheath 102c through the opening in the sheath guide wheel 108 and around the outer edge of the sheath guide wheel 108. In an embodiment, a back end of the catheter handle 102a of the catheter 102 may include a wired or wireless connector port 102b for connecting the catheter 102 to a processor of a catheter positioning system. In an embodiment, the sheath guide wheel 108 may include a grove along the outer edge to hold the catheter sheath 102c. The catheter sheath 102c may only partially encircle the outer edge of the sheath guide wheel 108 depending on the length of the catheter sheath 102c and an amount of sheath 102 extended or retracted in the C′ direction along the linear axis C. In some embodiments, the catheter sheath 102c may overlay itself in multiple turns around the sheath guide wheel 108, particularly when the sheath is significantly retracted.

The support frame 106 may include an introducer 106b configured to guide the catheter sheath 102c of the catheter 102 for insertion into a body of a patient. The support frame 106 may include a first roller 110a and second roller 110b. The first roller 110a and the second roller 110b may be configured such that the catheter sheath 102c bends around and moves freely across the first rollers 110a and the second roller 110b as the catheter sheath 102c is fed from the outer edge of the sheath guide wheel 108 to the introducer 106b. The circumference of the first roller 110a and second roller 110b may selected such that the bend in the catheter sheath 102c may not be too sharp as to cause damage to the catheter sheath 102c when being bent toward the introducer 106b and into the C′ direction along the linear axis C. Additionally, the circumference of the sheath guide wheel 108 may be selected such that the bend in the catheter sheath 102c as it is looped around the sheath guide wheel 108 may not be too sharp as to cause damage to the catheter sheath 102c. Further in the various embodiments, various portions of the catheter drive assembly 100 that interact with catheter 102, such as the catheter support 104, sheath guide wheel 108, rollers 110a, 110b, introducer 106b, etc., may be sterile components, either sterilizable or disposable, to avoid introducing contaminants into the body of a patient.

The rotation of the catheter support 104, sheath guide wheel 108, and catheter handle 102a clockwise or counterclockwise in the A′ direction about the axis of rotation A may cause the catheter sheath 102c of the catheter 102 held by the catheter support 104 to wind and unwind on the sheath guide wheel 108 and move (i.e., back or forth) in the C′ direction along the linear axis C, thereby extending or retracting the catheter sheath 102c through the introducer 106c. Rotation of the support frame 106 clockwise or counterclockwise in the B′ direction may rotate the support frame 106, sheath guide wheel 108, catheter support 104, and catheter handle 102a clockwise or counterclockwise in the B′ direction about the linear axis C, thereby rotating the catheter sheath 102c along its axis, which is generally the linear axis C. However, the catheter sheath 102c may follow an irregular path. Therefore, the catheter sheath 102c will rotate along and about its own axis. Regardless of the rotation of the sheath guide wheel 108 in the A′ direction about the axis of rotation A and/or the rotation of the support frame 106 in the B′ direction around the linear axis C, the catheter handle 102a and the catheter support 104 may not move forward or backward in the C′ direction along the linear axis C. In this manner, the catheter sheath 102c may be extended or retracted and/or rotated to position the catheter sheath 102c as needed within a patient while the catheter drive assembly 100 may remain linearly stationary by not moving in the C′ direction along the linear axis C.

To illustrate the rotation of the catheter drive assembly 100, referring to FIG. 2, the catheter drive assembly 100 is illustrated with the support frame 106 rotated 15 degrees in the B′ (e.g., clockwise) direction around the linear axis C and the sheath guide wheel 108 rotated 8 degrees in the A′ direction (e.g., counterclockwise) around the axis of rotation A. FIG. 2 illustrates that while the catheter support 104, catheter handle 102a, and sheath guide wheel 108 may rotate around the axis of rotation A, the frame support 106 may not and the introducer 106b may continue to align the catheter sheath 102c in the C′ direction along the linear axis C. Additionally, FIG. 2 illustrates that while the catheter support 104, catheter handle 102a, and sheath guide wheel 108 may rotate around the axis of rotation A, the catheter support 104, catheter handle 102a, and sheath guide wheel 108 may not move in the C′ direction along the linear axis C. Further, by rotating the sheath guide wheel 108 about the A axis, the catheter sheath 102c may be extended (e.g., outfeed) and retracted (e.g., infeed) along the linear axis C while the catheter support 104, catheter handle 102a and sheath guide wheel 108 may remain stationary along the linear axis C.

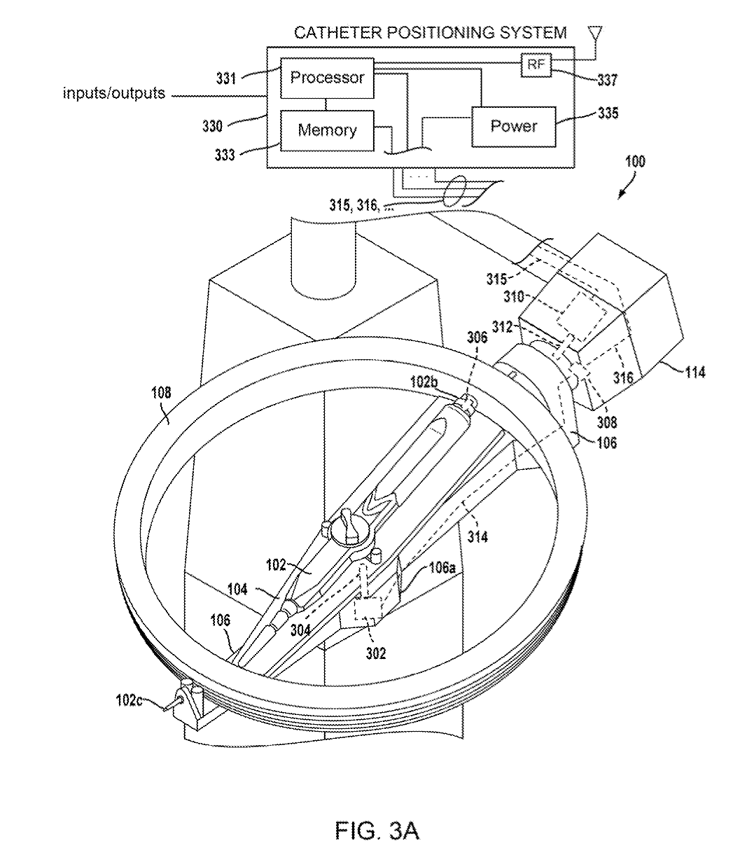

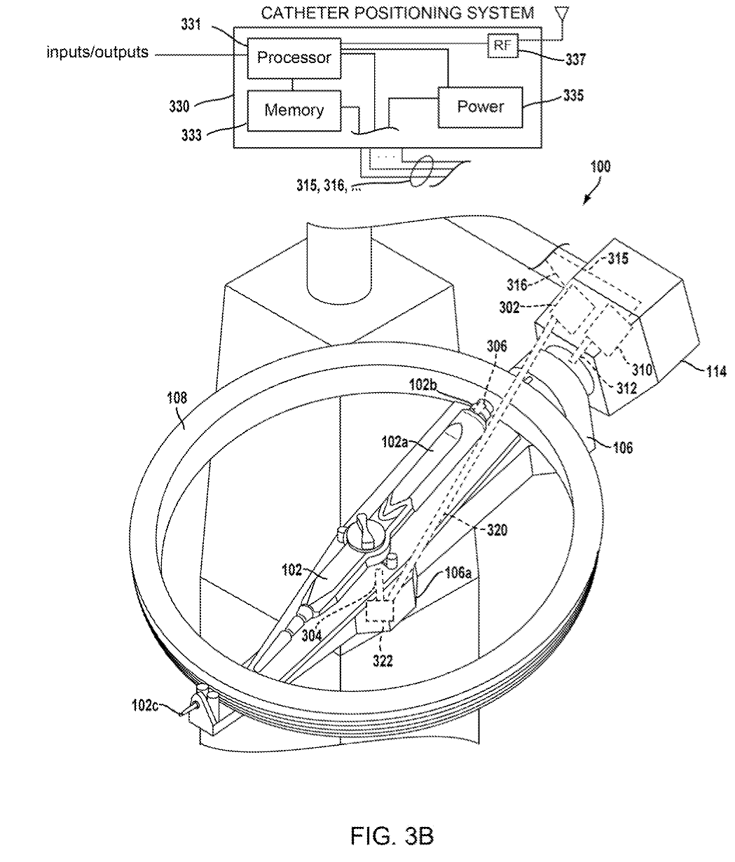

FIG. 3A illustrates internal components of the catheter drive assembly 100 described above with reference to FIG. 1 and FIG. 2 according to one or more embodiments. In some embodiments, the rotator housing 106a of the frame support 106 may include a guide wheel motor 302 coupled to the support frame 106 and housed or enclosed by the rotator housing 106a. The guide wheel motor 302 may be coupled to the shaft 304, which is coupled to and supports the catheter support 104, such as at a central rotational point. When actuated, the guide wheel motor 302 may rotate the shaft 304. The rotation of the shaft 304 may rotate the catheter support 104, sheath guide wheel 108, and catheter handle 102a about the shaft 304, such as about the central rotational point of the catheter support 104 to which the shaft 304 is coupled. A wire 314, or multiple wires 314, connected to the guide wheel motor 304 may pass through the frame support 106 to a rotating connector 308 (e.g., a slip ring, commutator, etc.), or multiple connectors 308, which may be connected to a wire 316, or multiple wires 316, in the drive box 114. The wire or wires 316 may connect to a processor of a catheter positioning system 330, which may be configured with a power source 335. The wire or wires 316 may provide control signals and/or may supply power to/from the guide wheel motor 302 via the wire or wires 316, rotating connector or connectors 308, and wire or wires 314. In this manner, a processor 331 of the catheter positioning system 330 may control the actuation of the guide wheel motor 302 and thereby control the extension or retraction of the catheter sheath 102c in the direction C′ along the linear axis C. As an example, the processor 331 of the catheter positioning system 330 may be configured with processor-executable instructions, which may be stored in a memory 333 or may be programmed directly into the processor 331 to perform operations to activate the guide wheel motor 302 in response to an input from a remote controller (not shown) connected to the processor 331.

In some embodiments, the drive box 114 may include a frame motor 310 coupled to the support frame 106. The frame motor 310 may be configured such that rotation of the shaft 312 of the frame motor 310 may rotate the support frame 106, sheath guide wheel 108, catheter support 104, and catheter handle 102a in the B′ direction about the linear axis C. In some embodiments, the frame motor 310 may be connected to a wire 315 or wires 315. The wire 315 or wires 315 may connect to the processor 331 of the catheter positioning system 330 and the power source 335, and control signals and power may thereby be provided to/from the frame motor 310 via the wire or wires 315. In this manner, the processor 331 of the catheter positioning system 330 may control the actuation of the frame motor 315 and may thereby control the rotation of the support frame 106, sheath guide wheel 108, catheter support 104, and catheter handle 102a in the direction B′ about the linear axis C. As an example, the processor 331 of the catheter positioning system 330 may be configured with processor-executable instructions, which may be stored in the memory 333 or programmed directly into the processor 331, to perform operations to activate the frame motor 315 in response to an input from a remote controller (not shown) connected to the processor 331.

In some embodiments, the connector port 102b may include a wireless transceiver 306 (e.g., a Bluetooth® transceiver) for connecting the catheter 102 to the processor 331 of a catheter positioning system, such as through an RF module 337. In this manner, the catheter 102 may wirelessly transmit and receive data and commands to and from the processor 331 of the catheter positioning system 330 either in addition to or as an alternative to wired connections 315, 316, etc. In this manner, wired connections from the catheter to the processor may be eliminated. However, power may be supplied via a wired connection. In other embodiments, the catheter 102 may be connected via one or more wire and rotating connector (e.g., one or more wire running from the connector port 102b through the catheter support 104, support frame 106, and/or drive box 114 with rotating connectors as needed to allow for rotations described above) to the processor 331 of the catheter positioning system 330.

FIG. 3B illustrates internal components of the catheter drive assembly 100 described above with reference to FIG. 1 and FIG. 2 according some embodiments. Although the illustrated embodiment may be similar to that illustrated in FIG. 3A, in the embodiment illustrated in FIG. 3B, the guide wheel motor 302 may be located in a portion of the catheter drive assembly 100 not supported by the support frame 106, such as the drive box 114. When the guide wheel motor 302 is located remote from the support frame 106, the guide wheel motor 302 may be coupled to a gear box 322 located in the rotator housing 106a of the frame support 106 which may be coupled to the shaft 304. The guide wheel motor 302 may be coupled to the gear box 322 by a drive shaft 320 extending from the drive box 114 through the support frame 106. The guide wheel motor 302 may rotate the drive shaft 320, which may in turn rotate one or more gears of the gear box 322 to rotate the shaft 304. With the guide wheel motor 302 located in the drive box 114, the wire 314 and rotating connector 308 shown in FIG. 3A may not be needed, and wire 316 may connect directly to the to the guide wheel motor 302.

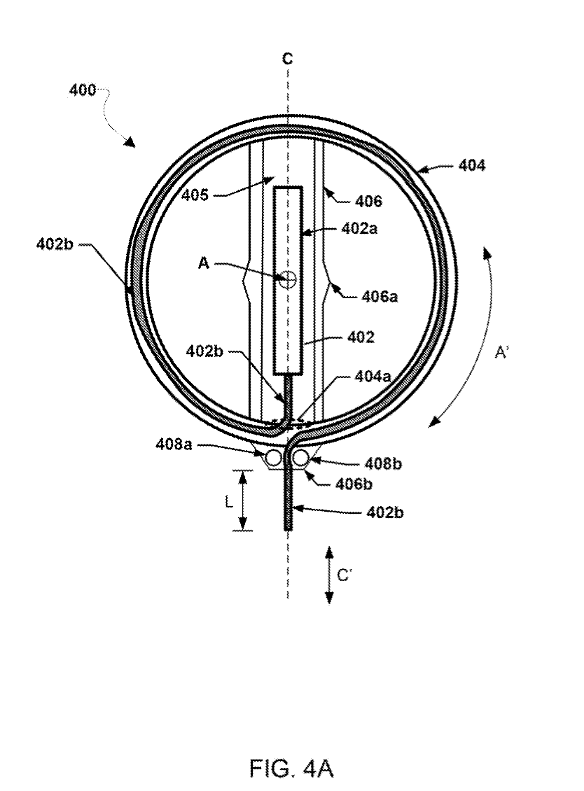

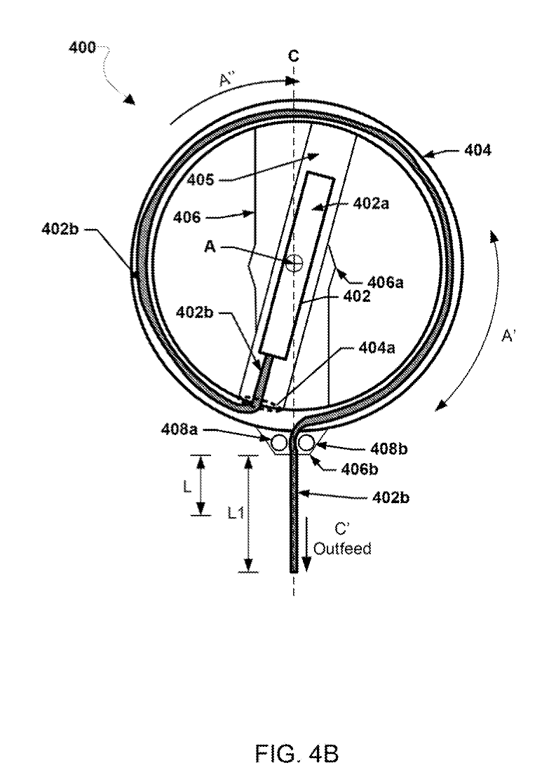

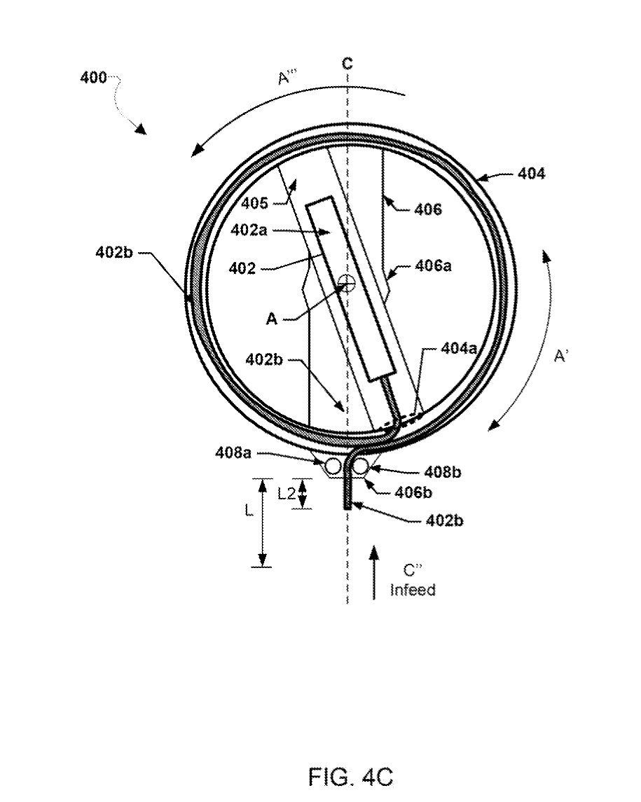

FIG. 4A through FIG. 4C illustrate translational movement between the rotational movement of an example loop drive 400 in the rotational direction A′ about the rotational axis A and the linear movement of a catheter sheath 402b in the linear direction C′ along the linear axis C. FIG. 4A illustrates the loop drive 400 in an initial position. The loop drive 400 may include a catheter support 405 that supports a handle 402a of a catheter 402. The catheter support 405 may be coupled to a sheath guide wheel 404. In the illustrated embodiment, the sheath guide wheel 404 may encircle the catheter support 405. In other embodiments, the sheath guide wheel 404 may be supported by the catheter support 405 without encircling the support. The catheter support 405 and sheath guide wheel 404 may be supported above a support frame 406. The support frame 406 may include a rotational housing 406a and an introducer 406b. The sheath 402b of the catheter 402 may extend from the catheter handle 402a and through an opening 404a in the sheath guide wheel 404. The sheath 402b may loop around an outer edge of the sheath guide wheel 404 in a direction, such as a clockwise direction. While a clockwise direction is illustrated, the sheath 402b may be wrapped in the counterclockwise direction. The sheath 402b may be wrapped around the perimeter of the sheath guide wheel 404 and may bend around rollers 408a and 408b such that the sheath 402b can extend away from the sheath guide wheel 404 and through the introducer 402b. The sheath guide wheel 404 may rotate in either direction of the arc A′ and the sheath 402b may extend or retract along the linear axis C. In the initial position illustrated in FIG. 4A, an example length L of section of sheath 402b may extend along the linear axis C from the introducer 406b. In some embodiments, the sheath guide wheel 404 may be sterile when in use, because it comes into contact with the sheath 402b. As discussed above, the guide wheel 108 may be removable so that after use it may be removed for disposal (i.e., the sheath guide wheel may be a single use component) or for sterilization prior to the next use. Other components, such as elements of the rotational housing (e.g., introducer 406b) may also be sterile (and disposable or resterilizable) as they may contact the catheter sheath.

FIG. 4B illustrates the loop drive 400 rotated to a second position from the initial position. The catheter support 405, catheter handle 402a, and sheath guide wheel 404 may have been rotated in a direction A″ along the rotational arc A′ about the A axis. The rotation of the sheath guide wheel 404 in the A″ direction may unspool the sheath 402b from around the sheath guide wheel 404. The rotation of the sheath guide wheel 404 in the A″ direction may extend an end of the sheath 402b in an outfeed direction C′ along the linear axis C to a new length L1 which may be farther from the introducer 406b than length L. At greater distances from the introducer 406b, the sheath 402b may take on curves and loops that cause those portions of the sheath 402b to be not straightly aligned with the linear axis C. However, the linear movement of the sheath 402b outward from the introducer 406b may be translated to linear movement along the entire length of the sheath 402b regardless of its localized shape. Thus, the outfeed of the catheter sheath 402b from the introducer 406b will move the tip of the catheter as well as any implements, irrigation hoses, or other objects a corresponding amount L1.

FIG. 4C illustrates the loop drive 400 rotated to a third position from the initial position. The catheter support 405, catheter handle 402a, and sheath guide wheel 404 may be rotated in a direction A′″ along the rotational arc A′ about the A axis. The rotation of the sheath guide wheel 404 in the A′″ direction may wind the sheath 402b around the sheath guide wheel 404. The rotation of the sheath guide wheel 404 in the A′″ direction may thereby retract the end of the sheath 402b in an infeed direction C″ along the linear axis C to a new length L2 which may be less than the length L. As with extending of the sheath 402b, the movement of the sheath guide wheel 404 in the A′″ direction may retract the sheath 402b along the entire length regardless of the localized shape of the sheath 402b, including any instruments coupled the sheath 402b and/or the catheter tip.

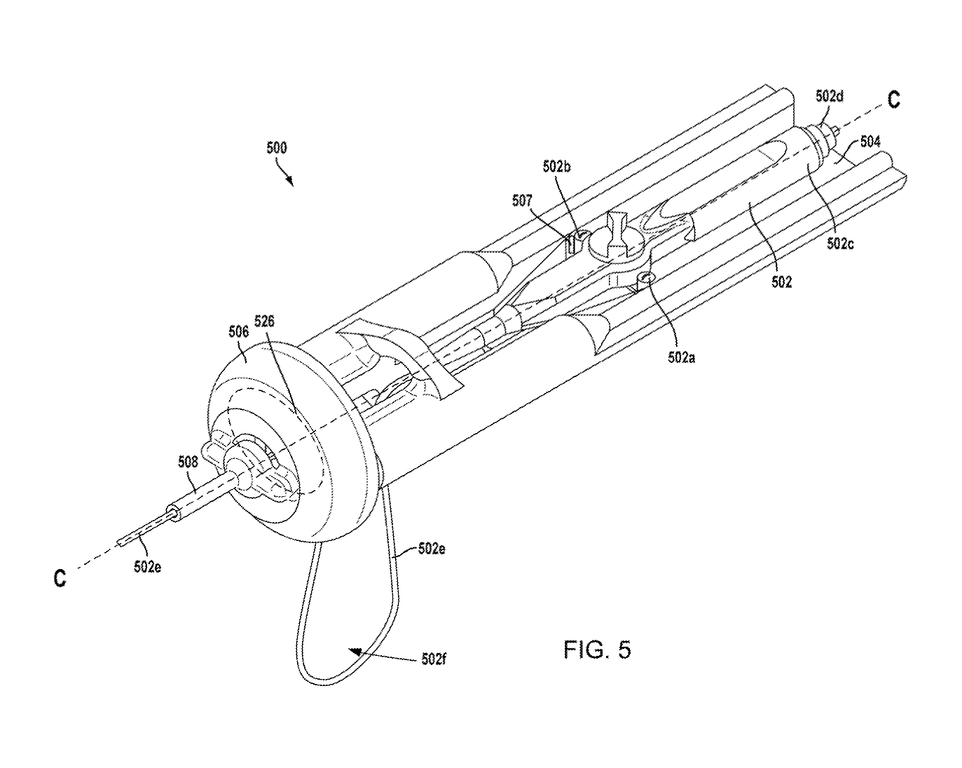

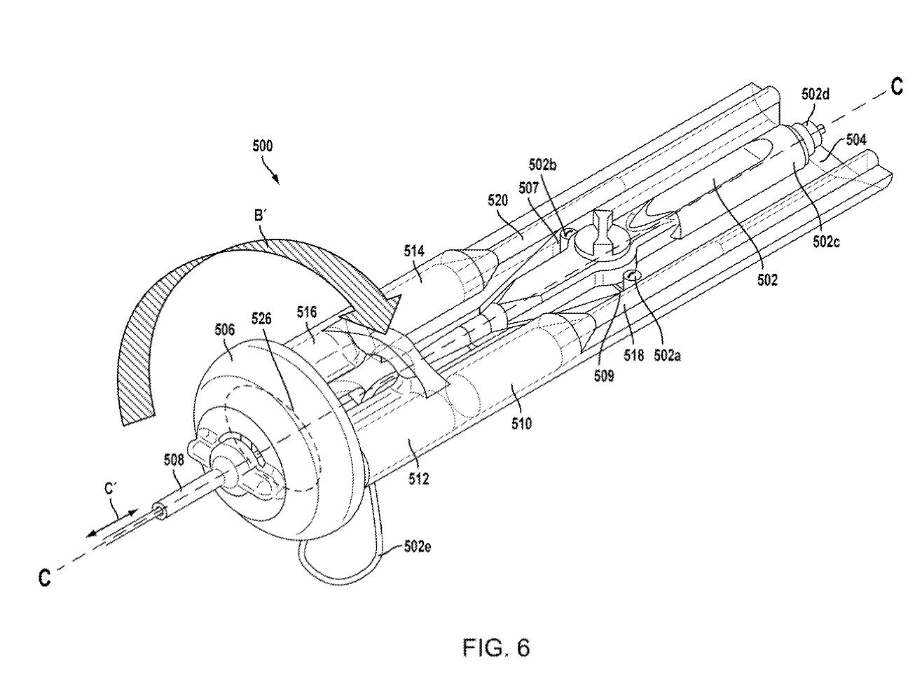

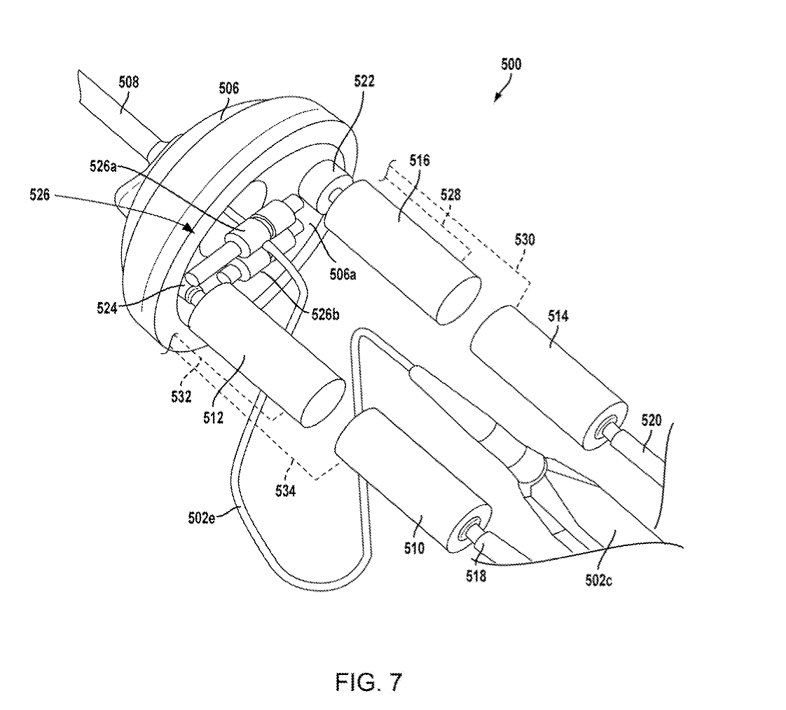

FIG. 5 through FIG. 7 illustrate a catheter drive assembly 500 including a pinch drive 526 according to embodiments. FIG. 5 illustrates various external components of the catheter drive assembly 500 from a front perspective view, FIG. 6 illustrates various external and internal components of the catheter drive assembly 500 from the front perspective view, and FIG. 7 illustrates various external and internal components of the catheter drive assembly 500 from a different perspective view.

Referring to FIG. 5, the catheter drive assembly 500 may include a catheter support 504 configured to hold a handle 502c of a catheter 502. The catheter 502 may include catheter controls 502a and 502b (e.g., rocker arms) which may interface with one or more control actuators 507 and 509 (see FIG. 6) on the catheter support 504. The control actuators 507 and 509 may move one or both of the catheter controls 502a and 502b to manipulate the catheter 502, such as by moving the control arms to cause a movement of a tip of the sheath 502e of the catheter 502. The sheath 502e of the catheter 502 may extend through a support frame 506 and connected introducer 508. The catheter support 504 and pinch drive unit 526 may be rotationally coupled to the support frame 506 such that support frame 506 and pinch drive unit 526 may rotate about the linear axis C. A length of sheath 502e not extended out the introducer along the linear axis C, may hang from an opening formed in the catheter support 504 in a loop 502f. In an embodiment, a back end of the handle 502c of the catheter 502 may include a wired or wireless connector port 502d for connecting the catheter 502 to a processor of a catheter positioning system (see, e.g., FIGS. 3A and 3B), thereby enabling the catheter 502 to send/receive data to/from the processor. In some embodiments, elements of the pinch drive unit 526 may be sterile when used because they may come in contact with the catheter 502. Such elements may be disposable or resterilizable.

Referring to FIG. 6, the catheter drive assembly 500 may include four motors coupled to the catheter support 504, including a roller motor 512, a frame motor 516, and two actuator motors 514 and 510. Each actuator motor 510 and 514 may be coupled to its own respective drive shaft 518 and 520. The drive shafts 518 and 520 may each interface with a respective control actuator 509 and 507. Actuation of the motor 510 and/or 514 may rotate the drive shaft 518 and/or 520, respectively, to actuate the control actuator 509 and/or 507. In an embodiment, actuation of the roller motor 512 may move the shaft 502e of the catheter 502 forward or backward in the direction C′ along the linear axis C. In an embodiment, actuation of the frame motor 516 may rotate the pinch drive unit 526, catheter support 504, and catheter handle 502c clockwise or counterclockwise in the direction B′ about the linear axis C.

Referring to FIG. 7, the pinch drive unit 526 of the catheter drive assembly 500 may include a first roller 526a and a second roller 526b. A gear or set of gears 524 may couple the roller motor 512 to the first roller 526a and the second roller 526b. As an example, a first gear of the set of gears 524 may interface with an end of the first roller 526a and a second gear may interface with the first gear of the set of gears 524 and an end of the second roller 526b. In this manner, the roller motor 512 may be activated to rotate the first roller 526a and the second roller 526b in opposite directions. The sheath 502e of the catheter 502 may extend between the first roller 526a and second roller 526b. The first roller 526a and second roller 526b may be configured to tightly contact or “pinch” the sheath 502e, such that rotation of the first roller 526a and second roller 526b in opposite directions moves the sheath 502e back and forth into/out of the introducer 508. The frame motor 516 may be coupled to a drive wheel 522 which may interface with an inner circumference 506a of the support frame 506. The rotation of the drive wheel 522 by the frame motor 516 may rotate the catheter support 504, roller motor 524, first roller 526a, second roller 526b, set of gears 524, frame motor 516, actuator motors 510, 514, catheter handle 502c, and other components supported by the catheter support 504 in the direction B′ about the linear axis C. In an embodiment, the first roller 526a and/or second roller 526b may be eccentric cams enabling the rate of insertion or extraction of the sheath 502e into/out of the introducer 508 to vary with the amount of rotation of the first roller 526a and/or second roller 526b. For example, a first profile of the eccentric cam may allow for faster insertion when the sheath 502e is first inserted into a patient and a second profile of the eccentric cam may allow for slower insertion when the sheath 502e is near a destination such as the heart of the patient. Various portions of the catheter drive assembly 500 that interact with a catheter 502, such as the catheter support 504, rollers 526a, 526b, introducer 508, etc., may be sterile components, either sterilizable or disposable, to avoid introducing contaminants into the body of a patient.

Additionally, FIG. 7 illustrates that the motors 510, 512, 514, and 516 may be connected to wires 534, 532, 530, and 528, respectively. The wires 534, 532, 530, and 528 may connect to a processor of a catheter positioning system and/or a power source, and control signals and/or power may be provide to/from the motors 510, 512, 514, and 516 via the wires 534, 532, 530, and 528, respectively such as illustrated and described in connection with FIG. 3A and FIG. 3B. As an example, the processor of the catheter positioning system may be configured with processor-executable instructions to perform operations to activate one or more of the motors 510, 512, 514, and 516 in response to one or more inputs from a remote controller connected to the processor.

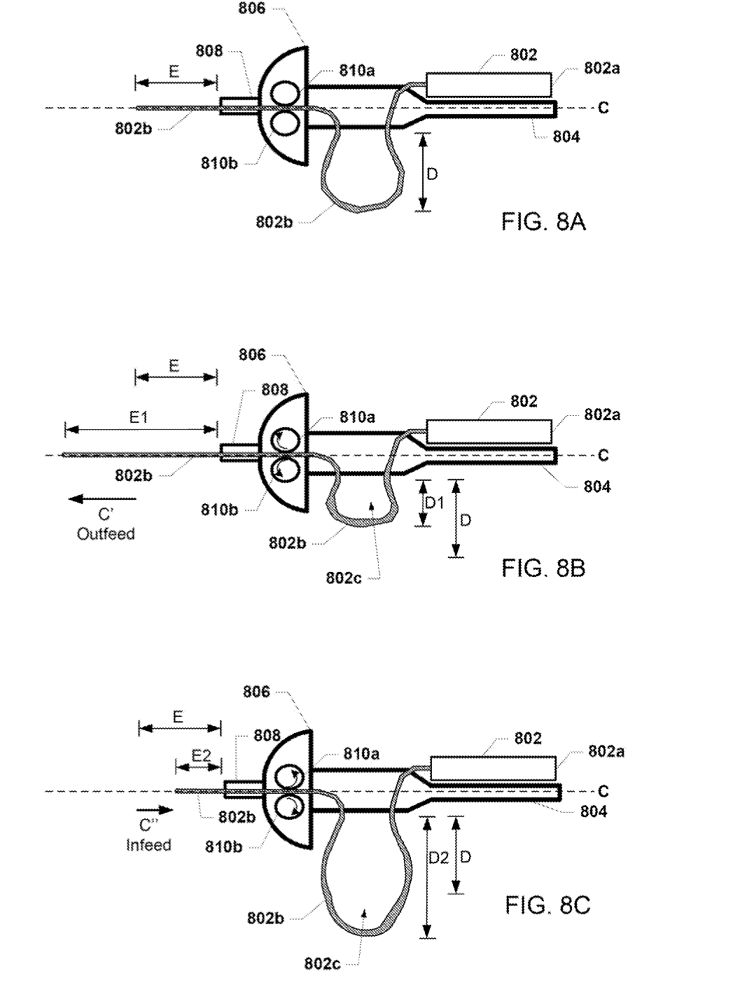

FIG. 8A through FIG. 8C illustrate translational movement between rotational movement of the rollers 810a and 810b of an example pinch drive and linear movement of a catheter sheath 802b in the direction C′ along the linear axis C. FIG. 8A illustrates the pinch drive in an initial position. The pinch drive may include a catheter support 804 configured to hold a handle 802a of a catheter 802, and a first roller 810a and a second roller 810b configured to rotate in opposite directions and to pinch the sheath 802b of the catheter 802. The sheath 806 may pass through the rollers 810a and 810b and may pass through an introducer 808 connected to the support frame 806. In the initial position illustrated in FIG. 8A, a length E of sheath 802b may extend along the linear axis C from the introducer 808 and a loop 802c of the sheath 802b may extend a distance D below the support frame 804. In some embodiments, the first roller 810a and the second roller 810b may be sterile when in use, due to the potential for contact with the catheter or catheter sheath. The first roller 810a and the second roller 810b may be removable for disposal after use or for sterilization prior to the next use.

FIG. 8B illustrates a second position in which the rollers 810a and 810b have rotated in opposite directions (e.g., roller 810a in a clockwise direction and 810b in a counterclockwise direction). The rotation and pinching action of the rollers 810a and 810b against the sheath 802b may cause the sheath 802b to move in an outfeed direction C′ from the introducer 808 along the linear axis C to a new length E1, which may be farther from the introducer 808 than length E. Additionally, the extension of the sheath 802b from the introducer 808 may reduce the length of the sheath 802b in the loop 802c below the catheter support 804, thereby reducing the loop 802c of the sheath 802b to a distance D1 below the support frame 804, which is shorter than the distance D.

FIG. 8C illustrates a third position in which the rollers 810a and 810b have rotated in opposite directions (e.g., roller 810a in a counterclockwise direction and 810b in a clockwise direction). The rotation and pinching action of the rollers 810a and 810b against the sheath 802b may cause an end of the sheath 802b to move in an infeed direction C″ from the introducer 808 along the linear axis C to a new length E2 which may be shorter than the length E, e.g. closer to the introducer 808. Additionally, the retraction of the sheath 802b toward the introducer 808 may increase the length of sheath 802b in the loop 802c below the catheter support 804, thereby increasing the loop 802c of the sheath 802b to a distance D2 below the support frame 804, which is longer than the distance D.

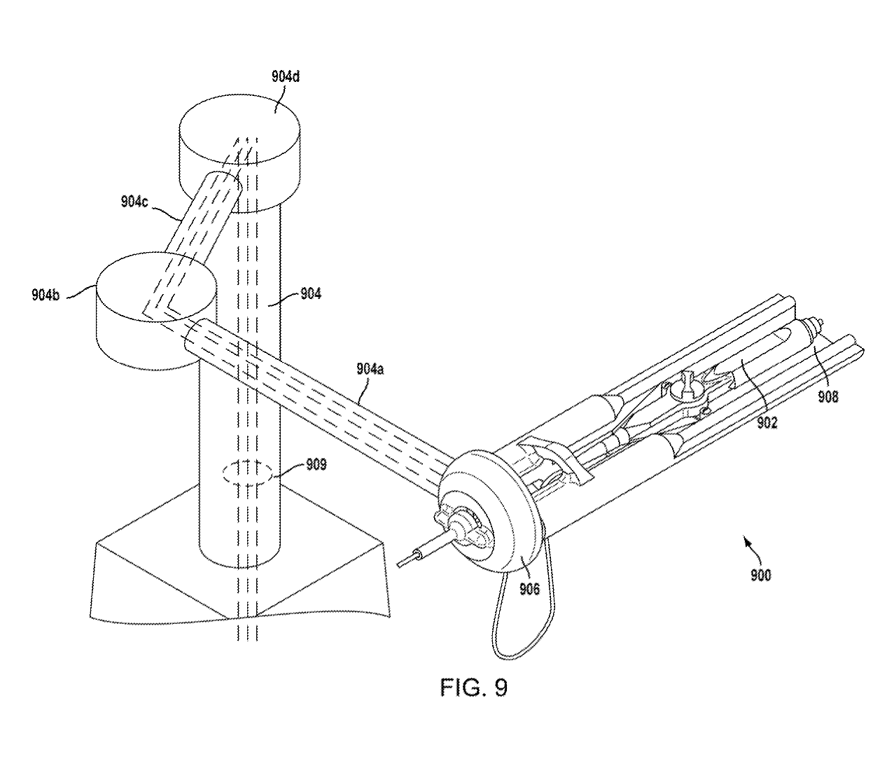

FIG. 9 illustrates a catheter drive assembly 900 (e.g., similar to catheter drive assembly 500 described above with reference to FIG. 5 through FIG. 7), which may be coupled to a base 904. The catheter drive assembly 900 may be coupled to a base 904 including various articulating joints 904b, 904d, and arms 904a, 904c. The arm 904a may be coupled to the support frame 906 of the catheter drive assembly 900. In an embodiment, wires 909 for connecting the catheter drive assembly 900 to power, data, etc., to/from the catheter drive assembly 900 (e.g., motor control signals, motor power, catheter data connections, etc.) may pass through the base 904 to the support frame 906. In an embodiment, the arm 904a of the base 904 may be rotationally coupled to the support frame 906 such that the angle of the catheter drive assembly 900 may be rotated about the arm 904a. In an embodiment, the arm 904a of the base 904 may hold the support frame 906 stationary while a frame motor rotates the catheter support 908 and handle of the catheter 902 about the linear axis.

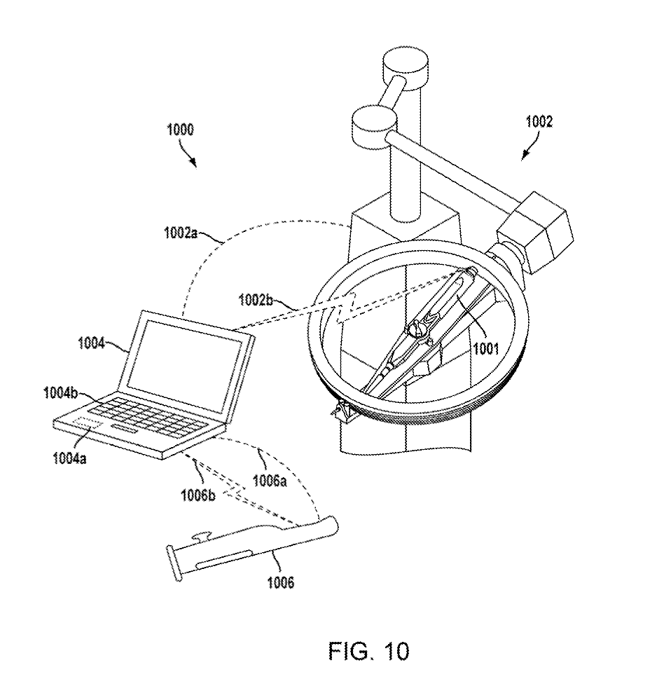

FIG. 10 is a system block diagram of an embodiment catheter positioning system 1000. FIG. 10 illustrates a loop drive type catheter drive assembly 1002 including a catheter 1001. While a loop drive type catheter drive assembly is illustrated, a pinch drive type catheter drive assembly may be substituted without changing the discussion of the operations of the catheter positioning system discussed below with reference to FIG. 10. A remote controller 1006 may be connected to a system processor 1004a of a programmable control system 1004 by one or more wired connectors 1006a or wireless data link 1006b. The system processor 1004a of the programmable control system 1004 may also be connected to the catheter drive assembly 1002 by one or more wired connector 1002a or wireless data link 1002b.

The system processor 1004a of the programmable control system 1004 may output control signals to actuate the motors of the catheter drive assembly 1002 based on inputs from the remote controller 1006. In some embodiments, the output control signals may also be based on training, calibration or programming routines, such as programmed movements for automatic positioning of the catheter 1001. Programmed movements of the catheter drive assembly 1002 and/or the catheter 1001 may be input prior to a medical procedure, such as by entering commands into the system processor of a programmable control system 1004 (e.g., via a keyboard 1004b) or by training the system, such as through manipulation of the remote controller 1006, such as during a training or calibration sequence. In particular, the processor 1004a of the programmable control system 1004 may be configured with processor-executable instructions to issue drive or power commands to each of the motors in the catheter drive assembly 1002 to control the relative rotations of each motor so as to move a catheter's sheath along a linear axis while holding the catheter handle stationary along that linear axis and/or rotating the catheter handle about the linear axis.

The system processor 1004a of the programmable control system 1004 may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but, in the alternative, the processor may be any conventional processor, controller, microcontroller, or state machine A processor may also be implemented as a combination of computing devices, e.g., a combination of a DSP and a microprocessor, a plurality of microprocessors, one or more microprocessors in conjunction with a DSP core, or any other such configuration. Alternatively, some operations may be performed by circuitry that is specific to a given function.

Those skilled in the art will recognize that the methods and systems of the present invention have many applications, may be implemented in many manners and, as such, are not to be limited by the preceding exemplary embodiments and examples. Additionally, the functionality of the components of the preceding embodiments may be implemented in different manners. Further, it is to be understood that the steps in the embodiments may be performed in any suitable order, combined into fewer steps or divided into more steps. Thus, the scope of the present invention covers conventionally known and future developed variations and modifications to the system components described herein, as would be understood by those skilled in the art.

What is claimed is:

1. A catheter drive assembly, comprising:
a catheter driver; and
a catheter support coupled to the catheter driver, the catheter support configured to hold a handle of a catheter,
wherein the catheter driver is configured to translate a rotational movement to a linear movement to move a sheath of the catheter along a linear axis while the catheter support and the handle of the catheter are stationary along the linear axis.

2. The catheter drive assembly of claim 1, wherein:
the catheter driver is a loop drive comprising:
a sheath guide wheel configured to hold at least a portion of the sheath of the catheter around an outer edge of the sheath guide wheel; and
a sheath guide wheel motor;
the catheter support is coupled to the loop drive such that the sheath guide wheel encircles the catheter support; and
the sheath guide wheel motor is configured to rotate the sheath guide wheel, catheter support, and handle of the catheter together about an axis of rotation other than the linear axis so as to move the sheath of the catheter along the linear axis.

3. The catheter drive assembly of claim 2, further comprising:
a support frame; and
a frame motor coupled to the support frame,
wherein:
the sheath guide wheel, the catheter support, and the catheter handle are supported by the support frame; and
the frame motor is configured to rotate the support frame, sheath guide wheel, catheter support, and catheter handle about the linear axis and to prevent the support frame, the sheath guide wheel, the catheter support, and the catheter handle from moving along the linear axis

4. The catheter drive assembly of claim 3, wherein at least a portion of the sheath guide wheel motor is supported by the support frame.

5. The catheter drive assembly of claim 3, wherein the sheath guide wheel motor is located in a portion of the catheter drive assembly not supported by the support frame.

6. The catheter drive assembly of claim 1, wherein the catheter driver is a pinch drive comprising:
a first roller;
a second roller, wherein the sheath of the catheter is pinched between the first roller and the second roller; and
a roller motor coupled to the first roller and the second roller,
wherein the roller motor is configured to rotate the first roller and the second roller in opposite directions to move the sheath of the catheter along the linear axis.

7. The catheter drive assembly of claim 6, further comprising a support frame, wherein:
the pinch drive is located within the support frame;
the pinch drive, the catheter support, and the catheter handle are supported by the support frame; and
the pinch drive and the catheter support are rotationally coupled to the support frame for rotation about the linear axis.

8. The catheter drive assembly of claim 7, further comprising a frame motor configured to rotate the pinch drive, the catheter support, and the catheter handle about the linear axis.

9. A catheter positioning system, comprising:
a catheter drive assembly, comprising:
a catheter driver; and
a catheter support coupled to the catheter driver, the catheter support configured to hold a handle of a catheter;
a processor; and
a remote controller coupled to the processor,
wherein the catheter driver is configured to move a sheath of the catheter along a linear axis while the catheter support and the handle of the catheter are stationary along the linear axis.

10. The catheter positioning system of claim 9, wherein:
the catheter driver is a loop drive comprising:
a sheath guide wheel configured to hold at least a portion of the sheath of the catheter around an outer edge of the sheath guide wheel; and
a sheath guide wheel motor;
the catheter support is coupled to the loop drive such that the sheath guide wheel encircles the catheter support;
the sheath guide wheel motor is configured to rotate the sheath guide wheel, the catheter support, and the handle of the catheter together about an axis of rotation other than the linear axis to move the sheath of the catheter along the linear axis; and
the processor is connected to the guide wheel motor and is configured with processor-executable instructions to actuate the guide wheel motor in response to an input from the remote controller.

11. The catheter positioning system of claim 10, wherein:
the catheter drive assembly further comprises:
a support frame; and
a frame motor coupled to the support frame;
the sheath guide wheel, the catheter support, and the catheter handle are supported by the support frame;
the frame motor is configured to rotate the support frame, the sheath guide wheel, the catheter support, and the catheter handle about the linear axis; and
the processor is connected to the frame motor and is configured with processor-executable instructions to actuate the frame motor in response to the input from the remote controller.

12. The catheter positioning system of claim 11, wherein at least a portion of the guide wheel motor is supported by the support frame.

13. The catheter positioning system of claim 11, wherein the guide wheel motor is located in a portion of the catheter drive assembly not supported by the support frame.

14. The catheter positioning system of claim 9, wherein:
the catheter driver is a pinch drive comprising:
a first roller;
a second roller, wherein the catheter sheath is pinched between the first roller and the second roller; and
a roller motor coupled to the first roller and the second roller;
the roller motor is configured to rotate the first roller and the second roller in opposite directions to move the sheath of the catheter along the linear axis; and
the processor is connected to the roller motor and is configured with processor-executable instructions to actuate the roller motor in response to an input from the remote controller.

15. The catheter positioning system of claim 14, wherein:
the catheter drive assembly further comprises a support frame,
the pinch drive is located within the support frame;
the pinch drive, the catheter support, and the catheter handle are supported by the support frame; and
the pinch drive and the catheter support are rotationally coupled to the support frame.

16. The catheter positioning system of claim 15, wherein:
the catheter drive assembly further comprises a frame motor;
the frame motor is configured to rotate the pinch drive, the catheter support, and the catheter handle about the linear axis; and
the processor is connected to frame motor and is configured with processor-executable instructions to actuate the frame motor in response to the input from the remote controller.

17. A catheter drive assembly, comprising:
a catheter driver configured to drive a catheter sheath along a linear axis; and
a catheter support coupled to the catheter driver, the catheter support configured to hold a handle of a catheter to which the catheter sheath is attached,
wherein:
the catheter driver comprises a rotational component contacting the catheter sheath, the rotational component configured to generate a rotational movement that is translated to a linear movement of the catheter sheath so as to drive the catheter sheath along a linear axis; and
the catheter support is configured to prevent movement of at least the catheter handle along the linear axis.

18. The catheter drive assembly of claim 17, wherein
the rotational component comprises a sheath guide wheel configured to hold at least a portion of the catheter sheath around an outer edge of the sheath guide wheel;
the catheter driver further comprises a sheath guide wheel motor to drive the sheath guide wheel; and
the sheath guide wheel motor is configured to rotate the sheath guide wheel, the catheter support, and the handle of the catheter together about an axis of rotation other than the linear axis so as to move the catheter sheath along the linear axis.

19. The catheter drive assembly of claim 17, wherein:
the rotational component comprises:
a first roller;
a second roller, wherein the catheter sheath is positioned in pinched relation between the first roller and the second roller; and
a roller motor coupled to the first roller and the second roller; and
the roller motor is configured to rotate the first roller and the second roller in opposite directions to move the catheter sheath along the linear axis.

20. A catheter positioning system, comprising:
a catheter drive assembly, comprising:
a catheter driver configured to drive a catheter sheath along a linear axis; and
a catheter support coupled to the catheter driver, the catheter support configured to hold a handle of a catheter to which the catheter sheath is attached;
a processor coupled to the catheter driver; and
a remote controller coupled to the processor,
wherein:
the catheter driver comprises a rotational component contacting the catheter sheath, the rotational component configured to generate a rotational movement that is translated to a linear movement of the catheter sheath so as to drive the catheter sheath along a linear axis under control of the processor based on input from the remote controller;
the catheter support is configured to prevent movement of at least the catheter handle along the linear axis.

21. The catheter positioning system of claim 20, wherein:
the catheter driver is a loop drive comprising:
a sheath guide wheel configured to hold at least a portion of the sheath of the catheter around an outer edge of the sheath guide wheel; and
a sheath guide wheel motor; and
the sheath guide wheel motor is configured to rotate the sheath guide wheel, the catheter support, and the handle of the catheter together about an axis of rotation other than the linear axis to move the sheath of the catheter along the linear axis under control of the processor based on the input from the remote controller.

22. The catheter positioning system of claim 20, wherein:
the catheter driver is a pinch drive comprising:
a first roller;
a second roller, wherein the catheter sheath is positioned in pinched relation between the first roller and the second roller; and
a roller motor coupled to the first roller and the second roller;
the roller motor is configured to rotate the first roller and the second roller in opposite directions to move the sheath of the catheter along the linear axis under control of the processor based on the input from the remote controller.
